(19) United States
(12) Patent Application Publication
(10) Pub. No.: US 2007/0286915 A1
(43) Pub. Date: Dec. 13, 2007

(54) Nerve Growth Factor Production Inhibitor and External Preparation for the Skin, Cosmetic, Quasi Drug, Preventive and Remedy for Atopic Dermatitis Containing the Nerve Growth Factor Production Inhibitor

(75) Inventors: Hiroshi Tonogaito, Hyogo (JP); Koichi Nakaoji, Hyogo (JP); Kaoru Sakai, Hyogo (JP); Kazuhiko Hamada, Kyoto (JP); Kenichi Nagamine, Tokyo (JP)

Correspondence Address: THE WEBB LAW FIRM, P.C., 700 KOPPERS BUILDING, 436 SEVENTH AVENUE, PITTSBURGH, PA, 15219, US

(73) Assignees: PIAS CORPORATION, Osaka-shi (JP); NICHIREI BIOSCHIENCES INC., Tokyo (JP)

(21) Appl. No.: 11/665,172

(22) PCT Filed: Oct. 14, 2005
(86) PCT No.: PCT/JP05/18947

§ 371 Date: Apr. 12, 2007

(30) Foreign Application Priority Data
Oct. 19, 2004 (JP) 2004-303956

(51) Int. Cl. A61K 36/185; A61K 131/00; A61P 17/00

(52) U.S. Cl. 424/776

(57) ABSTRACT

It is an object of the present invention to provide a nerve growth factor production inhibitor having a function to inhibit the production of the nerve growth factor deeply involved with the extension of C fiber to epidermis, as well as a high safety, and an external preparation for the skin, a cosmetic, a quasi drug, a preventive and remedy for itching and a remedy for atopic dermatitis containing the nerve growth factor production inhibitor. The nerve growth factor production inhibitor is characterized in that an acerola seed extract is combined.

### TECHNICAL FIELD

The present invention relates to a nerve growth factor production inhibitor and external preparation for the skin, cosmetic, quasi drug, preventive and remedy for itching and remedy for atopic dermatitis containing the nerve growth factor production inhibitor.

### RELATED ART

Itching is a typical sensation occurring only in skin, mucosa and cornea, and is explained as “an uneasy sensation accompanied by want for scratching”. In an inflammatory or allergic skin disorder, such as an atopic skin disorder, a pollenosis, a food allergy and an urticaria recently issued as a social problem, the itching sensation is one of very significant and serious symptoms. In addition, itching accompanied with a senile pruritus accompanied by drying of skin, an icterus or a dialysis and itching in a complication of diabetes and malignant tumor are also issued as serious problems.

The itching is caused as peripheral itching or central itching by various stimuli such as physical stimuli including drying of skin, temperature variation, sweat, compression, contact or the like and chemical stimuli by itching material, and the like. It is only histamine that is the most important material as an endogenous itching material, as well as its antagonist being clinically used. It is considered that H₁ acceptor is involved with this. An external preparation containing a competing antisubstance of histamine, for example, chlorpheniramine maleate, diphenhydramine and a related material thereto is used for treatment of the itching. However, it is considered to be a problem that an external preparation with the antihistamine action produces side effects.

Consequently, a patent application as the following Patent Document 1 related to the external preparation containing a natural component, in which safety is focused on is filed. The Patent Document 1 discloses the invention of an external preparation for the skin containing a bamboo extraction component. In the “Related Art” section, an external preparation utilizing mast cell membrane stabilizing ability of borneol (Japanese Patent Application Laid-Open No. Hei-06-211713), an external preparation utilizing inflammation inhibiting function by actinomyces culture fluid (Japanese Patent Application Laid-Open No. Hei-05-25053) and an external preparation utilizing antiallergy of fat and oil containing docosahexaenoic acid (DHA) or linolenic acid (Japanese Patent Application Laid-Open No. Hei-02-29081) are disclosed. It is also disclosed that these natural antiallergic drugs produce few side effects, but do not have sufficient effect. Patent Document 1: Japanese Patent Application Laid-Open No.2003-212786

However, even the invention of the Patent Document 1 could only show the itching inhibition effect in the histamine isolation inhibition experiment or leukotriene secretion inhibition experiment.

On the other hand, in a recent research, transfer fiber terminal (C fiber terminal) of the itching, of which existence is recognized only to the boundary of epidermis and dermis in healthy skin, extends to epidermis of skin having atopic dermatitis or dried skin. In this respect, it is pointed out that the extension of the C fiber terminal to epidermis is a cause of intense itching in senile pruritus, atopic dermatitis and dried skin.

These problems are disclosed in, for example, the “Related Art” section of the following Patent Document 2 (Paragraphs “0005” to “0007” in the specification). The invention of the Patent Document 2 is characterized in that an extract of flower petal of jewelweed is contained in an antiallergic composition. However, since it is not attempting to solve the problems with clarifying the mechanism of such itching, the effect of the invention is limited to antiallergic and antianaphylaxis effect. Patent Document 2: Japanese Patent Application Laid-Open No.2001-278796

At all events, as it stands, the itching inhibitor having a mechanism of action that inhibits the extension of C fiber to skin epidermis has not yet been developed.

### DISCLOSURE OF THE INVENTION

### Problems to be Solved by the Invention

The present invention has been conceived to solve the abovementioned problems. It is an object of the present invention to provide a nerve growth factor production inhibitor having the function to inhibit the production of the nerve growth factor deeply involved with the extension of C fiber to epidermis, as well as having high safety, and an external preparation for the skin, cosmetic, quasi drug, preventive and remedy for itching and remedy for atopic dermatitis containing the nerve growth factor production inhibitor.

### Means for Solving the Problems

As a result of the intensive researches performed to solve the problems, the present inventors have found that an acerola seed extract has an excellent nerve growth factor production inhibition function, especially, it eases and improves an inflammation or an itching produced in skin and is safe for a living body. Thus, the present invention has been achieved.

That is, the present invention is provided as a nerve growth factor production inhibitor, and external preparation for the skin, cosmetic, quasi drug, preventive and remedy for itching and remedy for atopic dermatitis containing the nerve growth factor production inhibitor. The invention recited in claim 1, relating to a nerve growth factor production inhibitor, is characterized in that an acerola seed extract is combined in the nerve growth factor production inhibitor.

In the present invention, by “an acerola seed extract is combined in the nerve growth factor production inhibitor” is meant that the nerve growth factor production inhibitor may include materials other than the acerola seed extract in addition to the material consisting only of acerola seed extract.

The invention recited in claim 2 relates to an external preparation for the skin in which the nerve growth factor production inhibitor of claim 1 is combined. The invention recited in claim 3 relates to a cosmetic in which the nerve growth factor production inhibitor of claim 1 is combined. The invention recited in claim 4 relates to a quasi drug in which the nerve growth factor production inhibitor of claim 1 is combined.

Further, the invention recited in claim 5 relates to preventive and remedy for itching in which the nerve growth factor production inhibitor of claim 1 is combined. The invention recited in claim 6 relates to remedy for atopic dermatitis in which the nerve growth factor production inhibitor of claim 1 is combined.

As described above, the C fiber terminal, of which existence is recognized only to the boundary of epidermis and dermis in healthy skin, extends to epidermis in skin having atopic dermatitis and dried skin. Therefore, the extension of the C fiber terminal to epidermis is considered to be a cause of the itching. It is assumed that the C fiber terminal extends to epidermis since a nerve growth factor NGF acts on the C fiber terminal. Accordingly, the production of the NGF can be inhibited by using the nerve growth factor production inhibitor of the present application, thereby enabling prevention of the extension of the C fiber terminal to epidermis.

### Effect of the Invention

As described above, according to the present invention, it is possible to directly remove the cause of the itching by inhibiting the production of NGF which is the nerve growth factor deeply involved with the extension of C fiber to epidermis. In addition, it is possible to provide the nerve growth factor production inhibitor with high safety, as well as the external preparation for the skin, cosmetic, and the like containing the nerve growth factor production inhibitor.

### BRIEF DESCRIPTION OF THE DRAWINGS

FIG. 1 is a graph of a nerve growth factor production inhibition experiment with the use of normal human dermal fibroblast (NHDF), in which a correlation of the density of acerola seed extract and the isolation quantity of NGF is shown.

FIG. 2 is a graph showing an inhibition effect to contact dermatitis in mouse.

FIG. 3 is a graph showing an itching inhibition effect with use of an NC/Nga mouse.

### BEST MODE FOR CARRYING OUT THE INVENTION

An extract used in the present invention intends to various solvent extracts obtained by extracting with a solvent at low temperature or room temperature to warming temperature, after crushing the seed of acerola (scientific name: Malpighia emarginata DC) which is either dried or not dried, or extracting with an extracting instrument such as a soxhlet extractor, a diluted solution thereof, a concentrated solution thereof or a dried powder thereof.

The extracting solvent can be obtained by one kind or combination of two or more kinds out of water, lower univalence alcohol such as methanol, ethanol and the like, liquid polyol such as glycerin, propylene glycol, 1,3-butylene glycol and the like, hydroalcohols and so on. A preferable example of extracting method includes a method in which extraction is carried out for one to five days at room temperature with use of ethanol having hydrous concentration of 20 to 80% by volume or 1,3-butylene glycol, and then the resultant is filtered.

The quantity of each nerve growth factor production inhibitor of an acerola seed extract combined in the external preparation for the skin, cosmetic and the like is not particularly limited, but the weight of a dry solid material (total weight in case of containing plural extracts) is preferably 0.0001 to 20.0% by weight on the basis of total weight. It is because when the combining quantity is less than 0.0001% by weight, the effect of the present invention is not sufficiently obtained, while even when the combining quantity is greater than 20.0% by weight, an improvement in the effect comparable to weight increase is not recognizable. In this sight, it is more preferable that the combining quantity is 0.0005 to 5.0% by weight.

The nerve growth factor production inhibitor of the present invention can be dosage form of, for example, lotions, emulsions, creams, ointments, facial masks, foundations and the like as the external preparation for the skin. A coloring matter, antiseptic agent, detergent, fragrant materials, pigment and the like can be appropriately combined in the nerve growth factor production inhibitor of the present invention in compliance with its form.

### EXAMPLES

Hereinafter, examples of the present invention are explained.

### Example 1

This example is an example of a nerve growth factor production inhibitor containing an acerola seed extract. A 1,3-butylene glycol aqueous solution of 140 kg containing 1,3-butylene glycol of 30% by weight was added to a crushed acerola seed of 80 kg, and the mixture was agitated at room temperature for a night. After centrifuging the total mixture, the supernatant thereof was filtered with a 0.22 μm filter to obtain the acerola seed extract. The quantity of the dry solid matter was 0.71% by weight.

### Experimental Example 1

This experimental example is a nerve growth factor production inhibition experiment using the normal human dermal fibroblast (NHDF). Specifically, the NHDF nerve growth factor production inhibition experiment was performed by using the nerve growth factor production inhibitor of the acerola seed extract of the Example 1.

The normal human dermal fibroblast (NHDF) was cultured in Dulbecco's MEM culture medium (prepared by Sigma) containing 10% fetal bovine serum (processed at 56° C. for 30 minutes) under 37° C., 5% CO₂ atmosphere. The NHDF was suspended in Dulbecco's MEM culture medium containing 10% fetal bovine serum (processed at 56° C. for 30 minutes) to reach 10⁵ cells/ml. The thus suspended solution was dispensed to 24 well microplates (prepared by IWAKI) by 1 ml per well.

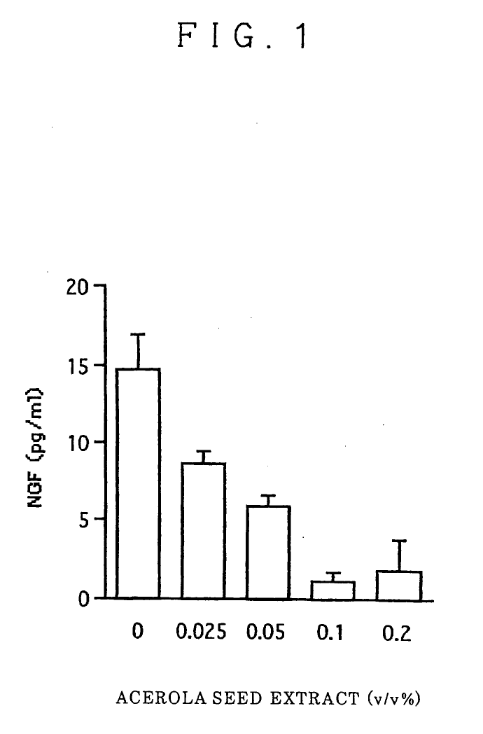

The supernatant was removed by suction after cultivation for 24 hours. Then, Dulbecco's MEM culture medium (prepared by Sigma) of 450 μl containing 10% cattle fetuses blood serum (processed at 56° C. for 30 minutes, prepared by ICN) containing preliminary prepared acerola seed extract of 0.2% by volume, 0.1% by volume, 0.05% by volume, or 0.025% by volume was dispensed. After incubation for 30 minutes, liquid solution of Dulbecco's MEM culture medium (prepared by Sigma) of 50 μl containing 100 ng/ml human interleukin 1α and 10% fetal bovine serum (processed at 56° C. for 30 minutes) was added, and then, incubation was made for another 24 hours. The culture supernatant was collected, and the concentration of the nerve growth factor therein was measured with NGF Emax Immunoassay kit (prepared by Promega). The measurement was represented by an average value and a standard deviation at N=4. The measurement result is shown in FIG. 1.

As being apparent from FIG. 1, the production of the nerve growth factor (the nerve growth factor concentration) in culture supernatant of fibroblast to which the acerola seed extract is not added was 14.69 pg/ml, while the nerve growth factor concentration in culture supernatant was 8.58 pg/ml in addition of 0.025% by volume, 5.87 pg/ml in addition of 0.05% by volume, and 1.09 pg/ml in addition of 0.1% by volume and 1.86 pg/ml in addition of 0.2% by volume when the acerola seed extract was added.

### Experimental Example 2

This experimental example is an experiment of the inhibition effect to mouse contact dermatitis. A 7-week-old BALB/c male mouse was purchased from SLC Co., Ltd. and was kept at room temperature of 23±3° C., humidity of 55±15%, and lighting cycle of 12 hours (light period of 7:00 to 19:00). The dosing (N=5) group of the acerola seed extract was set 30 minutes after sensitization. A 5% picrylchloride-ethanol solution of 0.15 ml was applied to abdominal skin of the mouse of which hair was cut on the day to perform the sensitization treatment. Then, after a lapse of 5 days, 1% solution of picrylchloride-acetone:olive oil (4:1) was applied to both sides of left pinna skin to bring about allergic reaction.

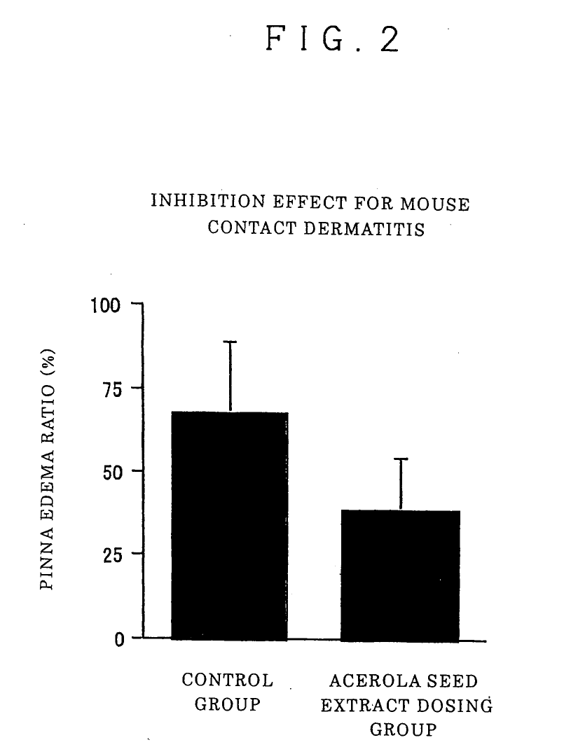

After the elapse of 30 minutes following the allergic reaction, a 50 μl acerola seed extract solution of 5% by volume prepared with ethanol solution of 50% by volume was dosed to the pinna portion of the mouse. As the control group, a 50 μl 1,3-butylene glycol solution of 1.5% by volume prepared with an ethanol solution of 50% by volume was dosed in the same manner. The thickness of the left pinna before the allergic reaction and after the elapse of 24 hours following the allergic reaction was measured by micrometer (MITUTOYO) so as to determine pinna edema inhibition ratio from pinna edema ratio. T-calibration was performed as the calibration of significant difference between the respective groups. The experiment result is shown in FIG. 2.

As being apparent from FIG. 2, the pinna edema ratio of the target group was 67.8±20.9%, while the pinna edema ratio of the acerola seed extract dosing group was 39.0±15.4% (p=0.035). The pinna edema was significantly inhibited by dosing the acerola seed extract. The above-mentioned experiment is used as the screening of atopic dermatitis and pollenosis.

### Experimental Example 3

This experiment example is the itching inhibition experiment using NC/Nga mouse. The NC/Nga mouse is a conventional grade animal which is a model mouse of the atopic dermatitis naturally developing the atopic dermatitis in which the itching is caused with the occurrence of the atopic dermatitis. Ten 4-week-old NC/Nga male mice were purchased from Nihon Charles River Co., Ltd. and were kept at room temperature of 23±3° C., humidity of 55±15%, and lighting cycle of 12 hours (light period of 7:00 to 19:00). Five mice were kept in each cage and those who developed atopic dermatitis through preliminary breeding were provided to the following experiment as five mice in per group. The group of continuous dosage (N=5) of the acerola seed extract for five days was set.

Hair on the mouse's back was shaved with a hair clipper and an electric shaver. A 150 μl acerola seed extract solution of 5% by volume prepared with an ethanol solution of 50% by volume was continuously applied to the mouse's back two times per day and five times per week. A 150 μl 1,3-butylene glycol solution of 1.5% by volume prepared with ethanol solution of 50% by volume was applied to the control group in the same manner. At the time the experiment was ended, the number of times at which respective mouse scratches their own back in 20 minutes was measured. The measured value was represented by average value and standard deviation. T-calibration was performed as the calibration of significant difference between the respective groups. The critical ratio of 5% or less was determined to be significant.

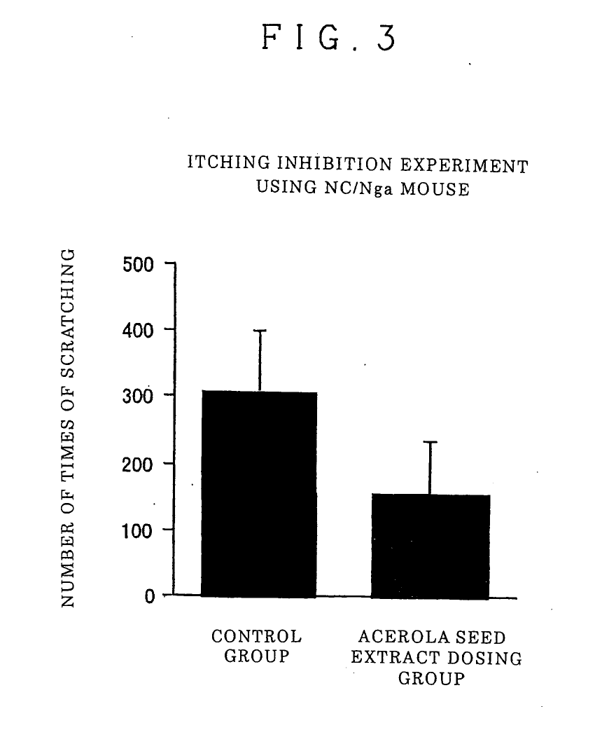

The experiment result is shown in FIG. 3. In the measurement of the number of times of scratching for 20 minutes at the time the experiment was ended, the control group shows 309±92 times and the acerola seed extract dosing group shows 158±79 times. The significant lowering in the number of times of scratching was recognized.

### Prescription Example 1

This prescription example is a prescription example of a cream as an example of the cosmetics. The composition of the prescription example is shown as follows.

| Composition | Combination ratio (% by weight) |
|---|---|
| Cetanol | 2.5% |
| Squalene | 10.0% |
| White beeswax | 1.0% |
| Glyceryltrioctanoate | 5.0% |
| Octyldodecylmyristate | 15.0% |
| Tocopherol acetate | 0.1% |
| 1,3-butylene glycol | 7.0% |
| Glyceryl monostearate | 3.0% |
| POE(20)sorbitan monostearate | 1.0% |
| Sorbitan monostearate | 2.0% |
| Acerola seed extract | 0.1% |
| Concentrated glycerin | 5.0% |
| Butyl parahydroxybenzoate | 0.1% |
| Ethyl parahydroxybenzoate | 0.2% |
| Purified water | Residual amount |

After heating and dissolving the cetanol, the squalene, the white beeswax and the octyldodecylmyristate among the above combination components, the glyceryltrioctanoate, the tocopherol acetate, the glyceryl monostearate, the POE (20) sorbitan monostearate (detergent) and the sorbitan monostearate are added, and evenly disperse and dissolve with the temperature adjusted to 70° C. Thus, an oily gel was obtained. Subsequently, the acerola seed extract, the 1,3-butylene glycol, the concentrated glycerin, the butyl parahydroxybenzoate (antiseptic agent), ethyl parahydroxybenzoate (antiseptic agent) are dissolved in purified water having a predetermined concentration. After adjusting the temperature to be 70° C., the thus obtained solution was slowly added to the oily gel with sufficiently agitating the same. After evenly mixed by a homo mixer, the solution was deaerated, filetered and cooled to 30° C., and thus, the cream was obtained.

### Prescription Example 2

This prescription example is also a prescription example of a cream. The composition of this example is the same with the Presecription Example 1 except in that the combining quantity of the acerola seed extract as the nerve growth factor production inhibitor is greater than that of Prescription Example 1. The composition thereof is shown as follows. The preparation of the cream was made in the same manner as Prescription Example 1.

| Composition | Combination ratio (% by weight) |
|---|---|
| Cetanol | 2.5% |
| Squalene | 10.0% |
| White beeswax | 1.0% |
| Glyceryltrioctanoate | 5.0% |
| Octyldodecylmyristate | 15.0% |
| Tocopherol acetate | 0.1% |
| 1,3-butylene glycol | 7.0% |
| Glyceryl monostearate | 3.0% |
| POE(20)sorbitan monostearate | 1.0% |
| Sorbitan monostearate | 2.0% |
| Acerola seed extract | 0.5% |
| Concentrated glycerin | 5.0% |
| Butyl parahydroxybenzoate | 0.1% |
| Ethyl parahydroxybenzoate | 0.2% |
| Purified water | Residual amount |

### Prescription Example 3

This prescription example is also a prescription example of a cream. The composition of this example is the same with the Presecription Examples 1 and 2 except in that the combining quantity of the acerola seed extract as the nerve growth factor production inhibitor is greater than that of Prescription Examples 1 and 2. The composition thereof is shown as follows. The preparation of the cream was made in the same manner as Prescription Example 1.

| Composition | Combination ratio (% by weight) |
|---|---|
| Cetanol | 2.5% |
| Squalene | 10.0% |
| White beeswax | 1.0% |
| Glyceryltrioctanoate | 5.0% |
| Octyldodecylmyristate | 15.0% |
| Tocopherol acetate | 0.1% |
| 1,3-butylene glycol | 7.0% |
| Glyceryl monostearate | 3.0% |
| POE(20)sorbitan monostearate | 1.0% |
| Sorbitan monostearate | 2.0% |
| Acerola seed extract | 1.0% |
| Concentrated glycerin | 5.0% |
| Butyl parahydroxybenzoate | 0.1% |
| Ethyl parahydroxybenzoate | 0.2% |
| Purified water | Residual amount |

### Experimental Example 4

In this experimental example, the itching inhibition experiment was performed for creams of Prescription Examples 1 to 3 described above. The experiment method is as follows. Creams of Prescription Examples 1 to 3 were continuously applied twice a day for 3 months to twenty five female examinees (25 years old to 45 years old) who complain about itching of skin accompanied with a dried skin. The evaluation is shown by the number of examinees answered that they no longer feel itching of skin. On the other hand, the same experiment was performed with a cream having the same cream base composition as Prescription Examples 1 to 3, in which the acerola seed extract was uncombined. The composition thereof is as follows.

(Comparative Example 1)

| Composition | Combination ratio (% by weight) |
|---|---|
| Cetanol | 2.5% |
| Squalene | 10.0% |
| White beeswax | 1.0% |
| Glyceryltrioctanoate | 5.0% |
| Octyldodecylmyristate | 15.0% |
| Tocopherol acetate | 0.1% |
| 1,3-butylene glycol | 7.0% |
| Glyceryl monostearate | 3.0% |
| POE(20)sorbitan monostearate | 1.0% |
| Sorbitan monostearate | 2.0% |
| Concentrated glycerin | 5.0% |
| Butyl parahydroxybenzoate | 0.1% |
| Ethyl parahydroxybenzoate | 0.2% |
| Purified water | Residual amount |

The experiment result is shown in Table 1.

TABLE 1

| | Comparative | Prescription | Prescription | Prescription |
|---|---|---|---|---|
| | Example 1 | Example 1 | Example 2 | Example 3 |
| Number of | 7 | 7 | 11 | 18 |
| Examinees | | | | |

As being apparent from Table 1, the number of examinees who answered that they no longer feel itching of skin in Prescription Example 1 was the same as that of the Comparative Example 1. However, the number of examinees who answered that they no longer feel itching of skin in Prescription Example 2 was 1.5 times greater than that of the Comparative Example 1. Further, the number of examinees who answered that they no longer feel itching of skin in Prescription Example 3 was more than about twice of the Comparative Example 1.

### Experimental Example 5

This experimental example is an experiment of inhibiting an inflammation caused by ultraviolet rays. The remedy effect of the inflammation was examined by using the creams of Prescription Examples 1 to 3 described above. On the other hand, the inteban cream (prepared by Sumitomo Pharmacy) which is a drug medicine containing one of the anti-inflammatory agents, indometacin, was used in Comparative Example 2 as the positive target.

### Experiment Method

A 4-week-old Hartley marmot was purchased from SLC Co., Ltd. and was kept at room temperature of 23±3° C., humidity of 55±15%, and lighting cycle of 12 hours (light period of 7:00 to 19:00). Hair on the marmot's back was shaved with a hair clipper and an electric shaver. Samples of Prescription Examples 1 to 3 and Comparative Examples 1 and 2 described in the above were continuously applied to a shaved part (2 cm×2 cm) for three days with the shaved part exposed to ultraviolet rays. The skin color of the part exposed to ultraviolet rays was measured with a colorimeter. The skin erythema was represented by a* value comparatively calculated by setting Comparative Example 1 as 100.

The experiment result is shown in Table 2.

TABLE 2

| | Com- | Com- | | | |
|---|---|---|---|---|---|
| | parative | parative | Prescription | Prescription | Prescription |
| | Example 1 | Example 2 | Example 1 | Example 2 | Example 3 |
| a* | 100 | 25 | 95 | 78 | 52 |
| value | | | | | |

As being apparent from FIG. 2, a* value in Prescription Example 1 has lowered a bit comparing to that in Comparative Example 1, however, a* value in Prescription Example 2 has lowered by about 20% comparing to that in Comparative Example 1. Furthermore, a* value in Prescription Example 3 has lowered to about a half of that in Comparative Example 1 but was not up to Comparative Example 2. However, it is generally known that an indometacin used in Comparative Example 2 has a side effect. Therefore, it is admissible that the creams in Prescription Examples 1 to 3 are superior in safety to that in Comparative Example 2.

### INDUSTRIAL AVAILABLENESS

The nerve growth factor production inhibitor of the present invention can be widely applied to external preparation, cosmetic, quasi drug, and the like. As far as use application is concerned, the nerve growth factor production inhibitor can also be widely used for remedy for atopic dermatitis, preventive and remedy for itching, compositions having a remedy function of skin problems caused by allergy, and the like.

1. A nerve growth factor production inhibitor, comprising an acerola seed extract.

2. An external preparation for skin, wherein the nerve growth factor production inhibitor according to claim 1 is combined.

3. A cosmetic, wherein the nerve growth factor production inhibitor according to claim 1 is combined.

4. A quasi drug, wherein the nerve growth factor production inhibitor according to claim 1 is combined.

5. A preventive and remedy for itching, wherein the nerve growth factor production inhibitor according to claim 1 is combined.

6. A remedy for atopic dermatitis, wherein the nerve growth factor production inhibitor according to claim 1 is combined.
